(19) United States
(12) Patent Application Publication
(10) Pub. No.: US 2023/0149455 A1
(43) Pub. Date: May 18, 2023

(54) ALKALINE WATER COMPOSITIONS AND USES THEREOF

(71) Applicant: G Water LLC, Waterford, MI (US)

(72) Inventors: Paul D. MANOS, Waterford, MI (US); Dean D. WESTON, Waterford, MI (US)

(21) Appl. No.: 17/905,615

(22) PCT Filed: Mar. 5, 2021
(86) PCT No.: PCT/US2021/021231

§ 371 Date: Sep. 2, 2022

Related U.S. Application Data
(60) Provisional application No. 62/986,109, filed on Mar. 6, 2020.

(51) Int. Cl. A61K 35/08; A61Q 5/02; A61Q 19/10; A61K 8/36; A61K 8/19; A61P 17/02

(52) U.S. Cl. CPC A61K 35/08; A61Q 5/02; A61Q 19/10; A61K 8/36; A61K 8/19; A61P 17/02

(57) ABSTRACT

Provided herein are alkaline water compositions and methods of using these compositions.

### CROSS-REFERENCE

This application claims benefit of U.S. Provisional Application No. 62/986,109, filed on Mar. 6, 2020, which is herein incorporated by reference in its entirety.

### BACKGROUND OF THE INVENTION

Alkaline water compositions are described herein that are useful in various health and beauty applications.

### SUMMARY OF THE INVENTION

Provided herein are alkaline water compositions comprising an aqueous solution of sodium propionate with no buffering agent. In one aspect, described herein is an alkaline water composition having the properties:

(a) about 200 mg/kg to about 1500 mg/kg of sodium;

(b) about 1000 mg/kg to about 5800 mg/kg of sodium as sodium propionate;

(c) total dissolved solids of about 1500 mg/L to about 5500 mg/L;

(d) pH of about 10.0 to about 13.5;

(e) total alkalinity as CaCO₃ of about 1100 mg/L to about 3000 mg/L;

(f) carbonate alkalinity as CaCO₃ of about 0 mg/L to about 1000 mg/L;

(g) bicarbonate alkalinity as CaCO₃ of about 1100 mg/L to about 2000 mg/L;

(h) oxidation reduction potential of about −150 to about 300; and

(i) electrical conductance of about 200 μmhos/cm to about 5800 μmhos/cm.

In another aspect, described herein is an alkaline water composition having the properties:

(a) about 1100 mg/kg to about 1500 mg/kg of sodium;

(b) about 5000 mg/kg to about 5800 mg/kg of sodium as sodium propionate;

(c) total dissolved solids of about 5000 mg/L to about 5500 mg/L;

(d) pH of about 10.0 to about 12.5;

(e) total alkalinity as CaCO₃ of about 1100 mg/L to about 3000 mg/L;

(f) carbonate alkalinity as CaCO₃ of about 0 mg/L to about 1000 mg/L;

(g) bicarbonate alkalinity as CaCO₃ of about 1100 mg/L to about 2000 mg/L;

(h) oxidation reduction potential of about −125 to about 300; and

(i) electrical conductance of about 3800 μmhos/cm to about 5800 μmhos/cm.

In some embodiments, the alkaline water composition has about 200 mg/kg to about 550 mg/kg of sodium. In some embodiments, the alkaline water composition has about 250 mg/kg to about 500 mg/kg of sodium. In some embodiments, the alkaline water composition has about 300 mg/kg to about 450 mg/kg of sodium. In some embodiments, the alkaline water composition has about 1200 mg/kg to about 1400 mg/kg of sodium. In some embodiments, the alkaline water composition has about 1200 mg/kg to about 1300 mg/kg of sodium. In some embodiments, the alkaline water composition has about 1300 mg/kg to about 1400 mg/kg of sodium. In some embodiments, the alkaline water composition has about 1000 mg/kg to about 1800 mg/kg of sodium as sodium propionate. In some embodiments, the alkaline water composition has about 1000 mg/kg to about 1500 mg/kg of sodium as sodium propionate. In some embodiments, the alkaline water composition has about 1100 mg/kg to about 1300 mg/kg of sodium as sodium propionate. In some embodiments, the alkaline water composition has about 5100 mg/kg to about 5750 mg/kg of sodium as sodium propionate. In some embodiments, the alkaline water composition has about 5100 mg/kg to about 5300 mg/kg of sodium as sodium propionate. In some embodiments, the alkaline water composition has about 5600 mg/kg to about 5750 mg/kg of sodium as sodium propionate. In some embodiments, the alkaline water composition has total dissolved solids of about 1500 mg/L to about 2000 mg/L. In some embodiments, the alkaline water composition has total dissolved solids of about 1650 mg/L to about 1750 mg/L. In some embodiments, the alkaline water composition has total dissolved solids of about 5200 mg/L to about 5400 mg/L. In some embodiments, the alkaline water composition has total dissolved solids of about 5200 mg/L to about 5300 mg/L. In some embodiments, the alkaline water composition has total dissolved solids of about 5300 mg/L to about 5400 mg/L. In some embodiments, the alkaline water composition has a pH of about 11.5 to about 13.5. In some embodiments, the alkaline water composition has a pH of about 12.5 to about 13.5. In some embodiments, the alkaline water composition has a pH of about 13.0 to about 13.5. In some embodiments, the alkaline water composition has a pH of about 10.5 to about 12.5. In some embodiments, the alkaline water composition has a pH of about 11.5 to about 12.5. In some embodiments, the alkaline water composition has a pH of about 12.0 to about 12.5. In some embodiments, the alkaline water composition has total alkalinity as CaCO₃ of about 1500 mg/L to about 2600 mg/L. In some embodiments, the alkaline water composition has total alkalinity as CaCO₃ of about 1700 mg/L to about 2200 mg/L. In some embodiments, the alkaline water composition has total alkalinity as CaCO₃ of about 1700 mg/L to about 1950 mg/L. In some embodiments, the alkaline water composition has total alkalinity as CaCO₃ of about 2050 mg/L to about 2200 mg/L. In some embodiments, the alkaline water composition has carbonate alkalinity as CaCO₃ of about 0 mg/L to about 5 mg/L. In some embodiments, the alkaline water composition has carbonate alkalinity as CaCO₃ of about 750 mg/L to about 1000 mg/L. In some embodiments, the alkaline water composition has bicarbonate alkalinity as CaCO₃ of about 1200 mg/L to about 1900 mg/L. In some embodiments, the alkaline water composition has bicarbonate alkalinity as CaCO₃ of about 1200 mg/L to about 1400 mg/L. In some embodiments, the alkaline water composition has bicarbonate alkalinity as CaCO₃ of about 1700 mg/L to about 1900 mg/L. In some embodiments, the alkaline water composition has oxidation reduction potential of about −150 to about −25. In some embodiments, the alkaline water composition has oxidation reduction potential of about −150 to about −100. In some embodiments, the alkaline water composition has oxidation reduction potential of about −100 to about 250. In some embodiments, the alkaline water composition has oxidation reduction potential of about −100 to about −25. In some embodiments, the filler is microcrystalline cellulose. In some embodiments, the alkaline water composition has oxidation reduction potential of about 200 to about 250. In some embodiments, the alkaline water composition has electrical conductance of about 200 μmhos/cm to about 700 μmhos/cm. In some embodiments, the alkaline water composition has electrical conductance of about 200 μmhos/cm to about 400 μmhos/cm. In some embodiments, the alkaline water composition has electrical conductance of about 200 μmhos/cm to about 300 μmhos/cm. In some embodiments, the alkaline water composition has electrical conductance of about 3900 μmhos/cm to about 5700 μmhos/cm. In some embodiments, the alkaline water composition has electrical conductance of about 3900 μmhos/cm to about 4000 μmhos/cm. In some embodiments, the alkaline water composition has electrical conductance of about 5500 μmhos/cm to about 5700 μmhos/cm.

In some embodiments, described herein is an alkaline water composition having the properties:

(a) about 250 mg/kg to about 500 mg/kg of sodium;

(b) about 1100 mg/kg to about 1300 mg/kg of sodium as sodium propionate;

(c) total dissolved solids of about 1650 mg/L to about 1750 mg/L;

(d) pH of about 13.0 to about 13.5;

(e) total alkalinity as CaCO₃ of about 2050 mg/L to about 2200 mg/L;

(f) carbonate alkalinity as CaCO₃ of about 750 mg/L to about 1000 mg/L;

(g) bicarbonate alkalinity as CaCO₃ of about 1200 mg/L to about 1400 mg/L;

(h) oxidation reduction potential of about −150 to about −25; and

(i) electrical conductance of about 200 μmhos/cm to about 300 μmhos/cm.

In some embodiments, described herein is an alkaline water composition having the properties:

(a) about 400 mg/kg of sodium;

(b) about 1200 mg/kg of sodium as sodium propionate;

(c) total dissolved solids of about 1650 mg/L to about 1750 mg/L;

(d) pH of about 13.2;

(e) total alkalinity as CaCO₃ of about 2120 mg/L;

(f) carbonate alkalinity as CaCO₃ of about 860 mg/L;

(g) bicarbonate alkalinity as CaCO₃ of about 1260 mg/L;

(h) oxidation reduction potential of about −121; and

(i) electrical conductance of about 254 μmhos/cm.

In some embodiments, described herein is an alkaline water composition having the properties:

(a) about 1300 mg/kg to about 1400 mg/kg of sodium;

(b) about 5600 mg/kg to about 5750 mg/kg of sodium as sodium propionate;

(c) total dissolved solids of about 5300 mg/L to about 5400 mg/L;

(d) pH of about 11.5 to about 12.5;

(e) total alkalinity as CaCO₃ of about 2050 mg/L to about 2200 mg/L;

(f) carbonate alkalinity as CaCO₃ of about 750 mg/L to about 1000 mg/L;

(g) bicarbonate alkalinity as CaCO₃ of about 1200 mg/L to about 1400 mg/L;

(h) oxidation reduction potential of about −100 to about −25; and

(i) electrical conductance of about 5500 μmhos/cm to about 5700 μmhos/cm.

In some embodiments, described herein is an alkaline water composition having the properties:

(a) about 1360 mg/kg of sodium;

(b) about 5685 mg/kg of sodium as sodium propionate;

(c) total dissolved solids of about 5350 mg/L;

(d) pH of about 12.2;

(e) total alkalinity as CaCO₃ of about 2120 mg/L;

(f) carbonate alkalinity as CaCO₃ of about 860 mg/L;

(g) bicarbonate alkalinity as CaCO₃ of about 1260 mg/L;

(h) oxidation reduction potential of about −82; and

(i) electrical conductance of about 5600 μmhos/cm.

In some embodiments, the alkaline water composition contains no buffers. In some embodiments, the alkaline water composition comprises pharmaceutical grade water. In some embodiments, the alkaline water composition comprises United States Pharmacopeia (USP) grade sodium propionate. In some embodiments, the alkaline water composition comprises European Pharmacopoeia (Ph. Eur.) grade sodium propionate. In some embodiments, the alkaline water composition comprises United States Pharmacopeia (USP) grade and European Pharmacopoeia (Ph. Eur.) grade sodium propionate.

In some embodiments, the alkaline water composition further comprises alcohol. In some embodiments, the alkaline water composition comprises about 10% to about 95% alcohol by volume. In some embodiments, the alkaline water composition comprises about 25% to about 90% alcohol by volume. In some embodiments, the alkaline water composition comprises about 75% alcohol and about 25% alkaline water by volume. In some embodiments, the alkaline water composition comprises about 65% alcohol and about 35% alkaline water by volume. In some embodiments, the alkaline water composition comprises about 50% alcohol and about 50% alkaline water by volume. In some embodiments, the alkaline water composition comprises alcohol, wherein the alcohol is selected from isopropanol, ethanol, and methanol. In some embodiments, the alkaline water composition comprises alcohol, wherein the alcohol is isopropanol. In some embodiments, the alkaline water composition comprises alcohol, wherein the alcohol is ethanol. In some embodiments, the alkaline water composition comprises alcohol, wherein the alcohol is methanol.

In some embodiments, the freezing point of the alkaline water composition is between about −60° C. to about −10° C. In some embodiments, the freezing point of the alkaline water composition is between about −50° C. to about −15° C. In some embodiments, the freezing point of the alkaline water composition is between about −40° C. to about −20° C. In some embodiments, the freezing point of the alkaline water composition is between about −40° C. to about −30° C.

In some embodiments of the alkaline water composition described herein, the alkaline water composition does not illicit a dermal irritation or sensitization response.

In some embodiments, the alkaline water composition is organic. In some embodiments, the alkaline water composition has a negative ionic charge. In some embodiments, the alkaline water composition is an anti-oxidant. In some embodiments, the alkaline water composition is hypoallergenic. In some embodiments, the alkaline water composition is hypo-osmotic. In some embodiments, the alkaline water composition is safe for human oral consumption.

In another aspect, provided herein is a method of cleansing eyelashes in an individual in need thereof, comprising administering to the individual in need thereof an effective amount of an alkaline water composition described herein.

In another aspect, provided herein is a method of cleansing the hair in an individual in need thereof, comprising administering to the individual in need thereof an effective amount of an alkaline water composition described herein.

In another aspect, provided herein is a method of cleansing the skin from sweat, dirt, or a combination thereof, in an individual in need thereof, comprising administering to the individual in need thereof an effective amount of an alkaline water composition described herein.

In some embodiments, the cleansing is anti-microbial cleansing.

In another aspect, provided herein is a method of hydrating the skin in an individual in need thereof, comprising administering to the individual in need thereof an effective amount of an alkaline water composition described herein.

In another aspect, provided herein is a method of hydrating the eyelashes in an individual in need thereof, comprising administering to the individual in need thereof an effective amount of an alkaline water composition described herein.

In another aspect, provided herein is a method of hydrating the hair in an individual in need thereof, comprising administering to the individual in need thereof an effective amount of an alkaline water composition described herein.

In another aspect, provided herein is a method of treating sunburn in an individual in need thereof, comprising administering to the individual in need thereof an effective amount of an alkaline water composition described herein.

In another aspect, provided herein is a method of treating wounds in an individual in need thereof, comprising administering to the individual in need thereof an effective amount of an alkaline water composition described herein.

In another aspect, provided herein is a method of treating pain in an individual in need thereof, comprising administering to the individual in need thereof an effective amount of an alkaline water composition described herein.

In another aspect, provided herein is a method of treating inflammation in an individual in need thereof, comprising administering to the individual in need thereof an effective amount of an alkaline water composition described herein.

### DETAILED DESCRIPTION OF THE INVENTION

### Certain Terminology

The singular forms “a,” “an,” and “the” include plural referents unless the context clearly dictates otherwise. When ranges are used herein, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range varies between 1% and 15% of the stated number or numerical range.

The terms “formulation” and “composition,” as used herein, are used interchangeably and refer to a mixture of two or more compounds, elements, or molecules. In some embodiments, the terms “formulation” and “composition” may be used to refer to a mixture or solution of one or more compounds with a carrier or other excipients.

The terms “effective amount” or “therapeutically effective amount” as used herein, refer to a sufficient amount of a composition or a compound being administered which will relieve to some extent one or more of the symptoms of the condition being treated. The result can be reduction and/or alleviation of the signs, symptoms, or causes of a condition, or any other desired alteration of a biological system.

Alkaline Water Compositions

In some embodiments, the alkaline water compositions described herein have some similar features to plasma in that the majority of the composition is water and it contains both inorganic and organic salt, nutrients, and dissolved gasses. Additionally, in some embodiments the alkaline water compositions described herein have a high pH, exhibit alkaline properties, have a negative ionic charge, and are non-corrosive.

In some embodiments is an alkaline water composition having the properties:

(a) about 200 mg/kg to about 1500 mg/kg of sodium;

(b) about 1000 mg/kg to about 5800 mg/kg of sodium as sodium propionate;

(c) total dissolved solids of about 1500 mg/L to about 5500 mg/L;

(d) pH of about 10.0 to about 13.5;

(e) total alkalinity as CaCO₃ of about 1100 mg/L to about 3000 mg/L;

(f) carbonate alkalinity as CaCO₃ of about 0 mg/L to about 1000 mg/L;

(g) bicarbonate alkalinity as CaCO₃ of about 1100 mg/L to about 2000 mg/L;

(h) oxidation reduction potential of about −150 to about 300; and

(i) electrical conductance of about 200 μmhos/cm to about 5800 μmhos/cm.

In some embodiments is an alkaline water composition having the properties:

(a) about 1100 mg/kg to about 1500 mg/kg of sodium;

(b) about 5000 mg/kg to about 5800 mg/kg of sodium as sodium propionate;

(c) total dissolved solids of about 5000 mg/L to about 5500 mg/L;

(d) pH of about 10.0 to about 12.5;

(e) total alkalinity as CaCO₃ of about 1100 mg/L to about 3000 mg/L;

(f) carbonate alkalinity as CaCO₃ of about 0 mg/L to about 1000 mg/L;

(g) bicarbonate alkalinity as CaCO₃ of about 1100 mg/L to about 2000 mg/L;

(h) oxidation reduction potential of about −125 to about 300; and

(i) electrical conductance of about 3800 μmhos/cm to about 5800 μmhos/cm.

In some embodiments described herein, the alkaline water composition has about 200 mg/kg to about 1000 mg/kg of sodium. In some embodiments described herein, the alkaline water composition has about 200 mg/kg to about 900 mg/kg of sodium. In some embodiments described herein, the alkaline water composition has about 200 mg/kg to about 800 mg/kg of sodium. In some embodiments described herein, the alkaline water composition has about 200 mg/kg to about 750 mg/kg of sodium. In some embodiments described herein, the alkaline water composition has about 200 mg/kg to about 700 mg/kg of sodium. In some embodiments described herein, the alkaline water composition has about 200 mg/kg to about 650 mg/kg of sodium. In some embodiments described herein, the alkaline water composition has about 200 mg/kg to about 600 mg/kg of sodium. In some embodiments described herein, the alkaline water composition has about 200 mg/kg to about 550 mg/kg of sodium. In some embodiments described herein, the alkaline water composition has about 250 mg/kg to about 550 mg/kg of sodium. In some embodiments described herein, the alkaline water composition has about 200 mg/kg to about 500 mg/kg of sodium. In some embodiments described herein, the alkaline water composition has about 250 mg/kg to about 500 mg/kg of sodium. In some embodiments described herein, the alkaline water composition has about 200 mg/kg to about 450 mg/kg of sodium. In some embodiments described herein, the alkaline water composition has about 250 mg/kg to about 450 mg/kg of sodium. In some embodiments described herein, the alkaline water composition has about 200 mg/kg to about 400 mg/kg of sodium. In some embodiments described herein, the alkaline water composition has about 250 mg/kg to about 400 mg/kg of sodium. In some embodiments described herein, the alkaline water composition has about 200 mg/kg to about 300 mg/kg of sodium. In some embodiments described herein, the alkaline water composition has about 300 mg/kg to about 400 mg/kg of sodium. In some embodiments described herein, the alkaline water composition has about 350 mg/kg to about 450 mg/kg of sodium. In some embodiments described herein, the alkaline water composition has about 1150 mg/kg to about 1500 mg/kg of sodium. In some embodiments described herein, the alkaline water composition has about 1150 mg/kg to about 1450 mg/kg of sodium. In some embodiments described herein, the alkaline water composition has about 1200 mg/kg to about 1450 mg/kg of sodium. In some embodiments described herein, the alkaline water composition has about 1200 mg/kg to about 1400 mg/kg of sodium. In some embodiments described herein, the alkaline water composition has about 1200 mg/kg to about 1350 mg/kg of sodium. In some embodiments described herein, the alkaline water composition has about 1200 mg/kg to about 1300 mg/kg of sodium. In some embodiments, the alkaline water composition has about 1200 mg/kg to about 1250 mg/kg of sodium. In some embodiments, the alkaline water composition has about 1250 mg/kg to about 1400 mg/kg of sodium. In some embodiments, the alkaline water composition has about 1300 mg/kg to about 1400 mg/kg of sodium. In some embodiments, the alkaline water composition has about 200 mg/kg of sodium. In some embodiments, the alkaline water composition has about 250 mg/kg of sodium. In some embodiments, the alkaline water composition has about 300 mg/kg of sodium. In some embodiments, the alkaline water composition has about 350 mg/kg of sodium. In some embodiments, the alkaline water composition has about 400 mg/kg of sodium. In some embodiments, the alkaline water composition has about 450 mg/kg of sodium. In some embodiments, the alkaline water composition has about 500 mg/kg of sodium. In some embodiments, the alkaline water composition has about 550 mg/kg of sodium. In some embodiments, the alkaline water composition has about 600 mg/kg of sodium. In some embodiments, the alkaline water composition has about 650 mg/kg of sodium. In some embodiments, the alkaline water composition has about 700 mg/kg of sodium. In some embodiments, the alkaline water composition has about 750 mg/kg of sodium. In some embodiments, the alkaline water composition has about 800 mg/kg of sodium. In some embodiments, the alkaline water composition has about 850 mg/kg of sodium. In some embodiments, the alkaline water composition has about 900 mg/kg of sodium. In some embodiments, the alkaline water composition has about 950 mg/kg of sodium. In some embodiments, the alkaline water composition has about 1000 mg/kg of sodium. In some embodiments, the alkaline water composition has about 1050 mg/kg of sodium. In some embodiments, the alkaline water composition has about 1100 mg/kg of sodium. In some embodiments, the alkaline water composition has about 1150 mg/kg of sodium. In some embodiments, the alkaline water composition has about 1200 mg/kg of sodium. In some embodiments, the alkaline water composition has about 1250 mg/kg of sodium. In some embodiments, the alkaline water composition has about 1300 mg/kg of sodium. In some embodiments, the alkaline water composition has about 1350 mg/kg of sodium. In some embodiments, the alkaline water composition has about 1400 mg/kg of sodium. In some embodiments, the alkaline water composition has about 1450 mg/kg of sodium. In some embodiments, the alkaline water composition has about 1500 mg/kg of sodium.

In some embodiments, the alkaline water composition has about 1000 mg/kg to about 2000 mg/kg of sodium as sodium propionate. In some embodiments, the alkaline water composition has about 1000 mg/kg to about 1800 mg/kg of sodium as sodium propionate. In some embodiments, the alkaline water composition has about 1000 mg/kg to about 1700 mg/kg of sodium as sodium propionate. In some embodiments, the alkaline water composition has about 1000 mg/kg to about 1650 mg/kg of sodium as sodium propionate. In some embodiments, the alkaline water composition has about 1000 mg/kg to about 1600 mg/kg of sodium as sodium propionate. In some embodiments, the alkaline water composition has about 1000 mg/kg to about 1550 mg/kg of sodium as sodium propionate. In some embodiments, the alkaline water composition has about 1000 mg/kg to about 1500 mg/kg of sodium as sodium propionate. In some embodiments, the alkaline water composition has about 1000 mg/kg to about 1450 mg/kg of sodium as sodium propionate. In some embodiments, the alkaline water composition has about 1050 mg/kg to about 1450 mg/kg of sodium as sodium propionate. In some embodiments, the alkaline water composition has about 1000 mg/kg to about 1400 mg/kg of sodium as sodium propionate. In some embodiments, the alkaline water composition has about 1050 mg/kg to about 1400 mg/kg of sodium as sodium propionate. In some embodiments, the alkaline water composition has about 1000 mg/kg to about 1350 mg/kg of sodium as sodium propionate. In some embodiments, the alkaline water composition has about 1050 mg/kg to about 1350 mg/kg of sodium as sodium propionate. In some embodiments, the alkaline water composition has about 1050 mg/kg to about 1300 mg/kg of sodium as sodium propionate. In some embodiments, the alkaline water composition has about 1100 mg/kg to about 1300 mg/kg of sodium as sodium propionate. In some embodiments, the alkaline water composition has about 1050 mg/kg to about 1250 mg/kg of sodium as sodium propionate. In some embodiments, the alkaline water composition has about 1100 mg/kg to about 1250 mg/kg of sodium as sodium propionate. In some embodiments, the alkaline water composition has about 1150 mg/kg to about 1250 mg/kg of sodium as sodium propionate. In some embodiments, the alkaline water composition has about 5050 mg/kg to about 5800 mg/kg of sodium as sodium propionate. In some embodiments, the alkaline water composition has about 5050 mg/kg to about 5750 mg/kg of sodium as sodium propionate. In some embodiments, the alkaline water composition has about 5100 mg/kg to about 5750 mg/kg of sodium as sodium propionate. In some embodiments, the alkaline water composition has about 5150 mg/kg to about 5750 mg/kg of sodium as sodium propionate. In some embodiments, the alkaline water composition has about 5150 mg/kg to about 5700 mg/kg of sodium as sodium propionate. In some embodiments, the alkaline water composition has about 5050 mg/kg to about 5350 mg/kg of sodium as sodium propionate. In some embodiments, the alkaline water composition has about 5100 mg/kg to about 5350 mg/kg of sodium as sodium propionate. In some embodiments, the alkaline water composition has about 5100 mg/kg to about 5300 mg/kg of sodium as sodium propionate. In some embodiments, the alkaline water composition has about 5100 mg/kg to about 5250 mg/kg of sodium as sodium propionate. In some embodiments, the alkaline water composition has about 5150 mg/kg to about 5250 mg/kg of sodium as sodium propionate. In some embodiments, the alkaline water composition has about 5100 mg/kg to about 5200 mg/kg of sodium as sodium propionate. In some embodiments, the alkaline water composition has about 5150 mg/kg to about 5200 mg/kg of sodium as sodium propionate. In some embodiments, the alkaline water composition has about 5500 mg/kg to about 5800 mg/kg of sodium as sodium propionate. In some embodiments, the alkaline water composition has about 5550 mg/kg to about 5800 mg/kg of sodium as sodium propionate. In some embodiments, the alkaline water composition has about 5550 mg/kg to about 5750 mg/kg of sodium as sodium propionate. In some embodiments, the alkaline water composition has about 5600 mg/kg to about 5750 mg/kg of sodium as sodium propionate. In some embodiments, the alkaline water composition has about 5650 mg/kg to about 5750 mg/kg of sodium as sodium propionate. In some embodiments, the alkaline water composition has about 5650 mg/kg to about 5700 mg/kg of sodium as sodium propionate. In some embodiments, the alkaline water composition has about 1000 mg/kg of sodium as sodium propionate. In some embodiments, the alkaline water composition has about 1050 mg/kg of sodium as sodium propionate. In some embodiments, the alkaline water composition has about 1100 mg/kg of sodium as sodium propionate. In some embodiments, the alkaline water composition has about 1150 mg/kg of sodium as sodium propionate. In some embodiments, the alkaline water composition has about 1200 mg/kg of sodium as sodium propionate. In some embodiments, the alkaline water composition has about 1250 mg/kg of sodium as sodium propionate. In some embodiments, the alkaline water composition has about 1300 mg/kg of sodium as sodium propionate. In some embodiments, the alkaline water composition has about 1350 mg/kg of sodium as sodium propionate. In some embodiments, the alkaline water composition has about 1400 mg/kg of sodium as sodium propionate. In some embodiments, the alkaline water composition has about 1450 mg/kg of sodium as sodium propionate. In some embodiments, the alkaline water composition has about 1500 mg/kg of sodium as sodium propionate. In some embodiments, the alkaline water composition has about 1550 mg/kg of sodium as sodium propionate. In some embodiments, the alkaline water composition has about 1600 mg/kg of sodium as sodium propionate. In some embodiments, the alkaline water composition has about 1650 mg/kg of sodium as sodium propionate. In some embodiments, the alkaline water composition has about 1700 mg/kg of sodium as sodium propionate. In some embodiments, the alkaline water composition has about 1750 mg/kg of sodium as sodium propionate. In some embodiments, the alkaline water composition has about 1800 mg/kg of sodium as sodium propionate. In some embodiments, the alkaline water composition has about 5000 mg/kg of sodium as sodium propionate. In some embodiments, the alkaline water composition has about 5050 mg/kg of sodium as sodium propionate. In some embodiments, the alkaline water composition has about 5100 mg/kg of sodium as sodium propionate. In some embodiments, the alkaline water composition has about 5150 mg/kg of sodium as sodium propionate. In some embodiments, the alkaline water composition has about 5200 mg/kg of sodium as sodium propionate. In some embodiments, the alkaline water composition has about 5250 mg/kg of sodium as sodium propionate. In some embodiments, the alkaline water composition has about 5300 mg/kg of sodium as sodium propionate. In some embodiments, the alkaline water composition has about 5350 mg/kg of sodium as sodium propionate. In some embodiments, the alkaline water composition has about 5400 mg/kg of sodium as sodium propionate. In some embodiments, the alkaline water composition has about 5450 mg/kg of sodium as sodium propionate. In some embodiments, the alkaline water composition has about 5500 mg/kg of sodium as sodium propionate. In some embodiments, the alkaline water composition has about 5550 mg/kg of sodium as sodium propionate. In some embodiments, the alkaline water composition has about 5600 mg/kg of sodium as sodium propionate. In some embodiments, the alkaline water composition has about 5650 mg/kg of sodium as sodium propionate. In some embodiments, the alkaline water composition has about 5700 mg/kg of sodium as sodium propionate. In some embodiments, the alkaline water composition has about 5750 mg/kg of sodium as sodium propionate. In some embodiments, the alkaline water composition has about 5800 mg/kg of sodium as sodium propionate.

In some embodiments, the alkaline water composition has total dissolved solids of about 1500 mg/L to about 2500 mg/L. In some embodiments, the alkaline water composition has total dissolved solids of about 1500 mg/L to about 2250 mg/L. In some embodiments, the alkaline water composition has total dissolved solids of about 1500 mg/L to about 2000 mg/L. In some embodiments, the alkaline water composition has total dissolved solids of about 1500 mg/L to about 1950 mg/L. In some embodiments, the alkaline water composition has total dissolved solids of about 1550 mg/L to about 1950 mg/L. In some embodiments, the alkaline water composition has total dissolved solids of about 1500 mg/L to about 1900 mg/L. In some embodiments, the alkaline water composition has total dissolved solids of about 1550 mg/L to about 1900 mg/L. In some embodiments, the alkaline water composition has total dissolved solids of about 1500 mg/L to about 1850 mg/L. In some embodiments, the alkaline water composition has total dissolved solids of about 1550 mg/L to about 1850 mg/L. In some embodiments, the alkaline water composition has total dissolved solids of about 1600 mg/L to about 1850 mg/L. In some embodiments, the alkaline water composition has total dissolved solids of about 1600 mg/L to about 1800 mg/L. In some embodiments, the alkaline water composition has total dissolved solids of about 1600 mg/L to about 1750 mg/L. In some embodiments, the alkaline water composition has total dissolved solids of about 1650 mg/L to about 1750 mg/L. In some embodiments, the alkaline water composition has total dissolved solids of about 5000 mg/L to about 5500 mg/L. In some embodiments, the alkaline water composition has total dissolved solids of about 5050 mg/L to about 5500 mg/L. In some embodiments, the alkaline water composition has total dissolved solids of about 5050 mg/L to about 5450 mg/L. In some embodiments, the alkaline water composition has total dissolved solids of about 5100 mg/L to about 5450 mg/L. In some embodiments, the alkaline water composition has total dissolved solids of about 5100 mg/L to about 5400 mg/L. In some embodiments, the alkaline water composition has total dissolved solids of about 5150 mg/L to about 5400 mg/L. In some embodiments, the alkaline water composition has total dissolved solids of about 5200 mg/L to about 5400 mg/L. In some embodiments, the alkaline water composition has total dissolved solids of about 5200 mg/L to about 5300 mg/L. In some embodiments, the alkaline water composition has total dissolved solids of about 5200 mg/L to about 5250 mg/L. In some embodiments, the alkaline water composition has total dissolved solids of about 5250 mg/L to about 5300 mg/L. In some embodiments, the alkaline water composition has total dissolved solids of about 5300 mg/L to about 5400 mg/L. In some embodiments, the alkaline water composition has total dissolved solids of about 5300 mg/L to about 5350 mg/L. In some embodiments, the alkaline water composition has total dissolved solids of about 5350 mg/L to about 5400 mg/L. In some embodiments, the alkaline water composition has total dissolved solids of about 1500 mg/L. In some embodiments, the alkaline water composition has total dissolved solids of about 1550 mg/L. In some embodiments, the alkaline water composition has total dissolved solids of about 1600 mg/L. In some embodiments, the alkaline water composition has total dissolved solids of about 1650 mg/L. In some embodiments, the alkaline water composition has total dissolved solids of about 1700 mg/L. In some embodiments, the alkaline water composition has total dissolved solids of about 1750 mg/L. In some embodiments, the alkaline water composition has total dissolved solids of about 1800 mg/L. In some embodiments, the alkaline water composition has total dissolved solids of about 1850 mg/L. In some embodiments, the alkaline water composition has total dissolved solids of about 1900 mg/L. In some embodiments, the alkaline water composition has total dissolved solids of about 1950 mg/L. In some embodiments, the alkaline water composition has total dissolved solids of about 2000 mg/L. In some embodiments, the alkaline water composition has total dissolved solids of about 5000 mg/L. In some embodiments, the alkaline water composition has total dissolved solids of about 5050 mg/L. In some embodiments, the alkaline water composition has total dissolved solids of about 5100 mg/L. In some embodiments, the alkaline water composition has total dissolved solids of about 5150 mg/L. In some embodiments, the alkaline water composition has total dissolved solids of about 5200 mg/L. In some embodiments, the alkaline water composition has total dissolved solids of about 5250 mg/L. In some embodiments, the alkaline water composition has total dissolved solids of about 5300 mg/L. In some embodiments, the alkaline water composition has total dissolved solids of about 5350 mg/L. In some embodiments, the alkaline water composition has total dissolved solids of about 5400 mg/L. In some embodiments, the alkaline water composition has total dissolved solids of about 5450 mg/L. In some embodiments, the alkaline water composition has total dissolved solids of about 5500 mg/L.

In some embodiments, the alkaline water composition has pH of about 10.0 to about 13.5. In some embodiments, the alkaline water composition has pH of about 10.5 to about 13.5. In some embodiments, the alkaline water composition has pH of about 11.0 to about 13.5. In some embodiments, the alkaline water composition has pH of about 11.5 to about 13.5. In some embodiments, the alkaline water composition has pH of about 12.0 to about 13.5. In some embodiments, the alkaline water composition has pH of about 12.5 to about 13.5. In some embodiments, the alkaline water composition has pH of about 13.0 to about 13.5. In some embodiments, the alkaline water composition has pH of about 10.0 to about 12.5. In some embodiments, the alkaline water composition has pH of about 10.5 to about 12.5. In some embodiments, the alkaline water composition has pH of about 11.0 to about 12.5. In some embodiments, the alkaline water composition has pH of about 11.5 to about 12.5. In some embodiments, the alkaline water composition has pH of about 12.0 to about 12.5. In some embodiments, the alkaline water composition has pH of about 10.0. In some embodiments, the alkaline water composition has pH of about 10.5. In some embodiments, the alkaline water composition has pH of about 11.0. In some embodiments, the alkaline water composition has pH of about 11.5. In some embodiments, the alkaline water composition has pH of about 12.0. In some embodiments, the alkaline water composition has pH of about 12.5. In some embodiments, the alkaline water composition has pH of about 13.0. In some embodiments, the alkaline water composition has pH of about 13.5.

In some embodiments, the alkaline water composition has total alkalinity as CaCO₃ of about 1200 mg/L to about 3000 mg/L. In some embodiments, the alkaline water composition has total alkalinity as CaCO₃ of about 1200 mg/L to about 2900 mg/L. In some embodiments, the alkaline water composition has total alkalinity as CaCO₃ of about 1300 mg/L to about 2900 mg/L. In some embodiments, the alkaline water composition has total alkalinity as CaCO₃ of about 1300 mg/L to about 2800 mg/L. In some embodiments, the alkaline water composition has total alkalinity as CaCO₃ of about 1400 mg/L to about 2800 mg/L. In some embodiments, the alkaline water composition has total alkalinity as CaCO₃ of about 1400 mg/L to about 2700 mg/L. In some embodiments, the alkaline water composition has total alkalinity as CaCO₃ of about 1500 mg/L to about 2700 mg/L. In some embodiments, the alkaline water composition has total alkalinity as CaCO₃ of about 1500 mg/L to about 2600 mg/L. In some embodiments, the alkaline water composition has total alkalinity as CaCO₃ of about 1600 mg/L to about 2600 mg/L. In some embodiments, the alkaline water composition has total alkalinity as CaCO₃ of about 1600 mg/L to about 2500 mg/L. In some embodiments, the alkaline water composition has total alkalinity as CaCO₃ of about 1650 mg/L to about 2500 mg/L. In some embodiments, the alkaline water composition has total alkalinity as CaCO₃ of about 1650 mg/L to about 2400 mg/L. In some embodiments, the alkaline water composition has total alkalinity as CaCO₃ of about 1700 mg/L to about 2400 mg/L. In some embodiments, the alkaline water composition has total alkalinity as CaCO₃ of about 1700 mg/L to about 2300 mg/L. In some embodiments, the alkaline water composition has total alkalinity as CaCO₃ of about 1700 mg/L to about 2200 mg/L. In some embodiments, the alkaline water composition has total alkalinity as CaCO₃ of about 1700 mg/L to about 2150 mg/L. In some embodiments, the alkaline water composition has total alkalinity as CaCO₃ of about 1700 mg/L to about 2100 mg/L. In some embodiments, the alkaline water composition has total alkalinity as CaCO₃ of about 1700 mg/L to about 2050 mg/L. In some embodiments, the alkaline water composition has total alkalinity as CaCO₃ of about 1700 mg/L to about 2000 mg/L. In some embodiments, the alkaline water composition has total alkalinity as CaCO₃ of about 1700 mg/L to about 1950 mg/L. In some embodiments, the alkaline water composition has total alkalinity as CaCO₃ of about 1750 mg/L to about 1950 mg/L. In some embodiments, the alkaline water composition has total alkalinity as CaCO₃ of about 1800 mg/L to about 1950 mg/L. In some embodiments, the alkaline water composition has total alkalinity as CaCO₃ of about 1800 mg/L to about 1900 mg/L. In some embodiments, the alkaline water composition has total alkalinity as CaCO₃ of about 1850 mg/L to about 1900 mg/L. In some embodiments, the alkaline water composition has total alkalinity as CaCO₃ of about 1800 mg/L to about 2500 mg/L. In some embodiments, the alkaline water composition has total alkalinity as CaCO₃ of about 1800 mg/L to about 2400 mg/L. In some embodiments, the alkaline water composition has total alkalinity as CaCO₃ of about 1800 mg/L to about 2300 mg/L. In some embodiments, the alkaline water composition has total alkalinity as CaCO₃ of about 1900 mg/L to about 2300 mg/L. In some embodiments, the alkaline water composition has total alkalinity as CaCO₃ of about 2000 mg/L to about 2300 mg/L. In some embodiments, the alkaline water composition has total alkalinity as CaCO₃ of about 2000 mg/L to about 2250 mg/L. In some embodiments, the alkaline water composition has total alkalinity as CaCO₃ of about 2050 mg/L to about 2250 mg/L. In some embodiments, the alkaline water composition has total alkalinity as CaCO₃ of about 2050 mg/L to about 2200 mg/L. In some embodiments, the alkaline water composition has total alkalinity as CaCO₃ of about 2050 mg/L to about 2150 mg/L. In some embodiments, the alkaline water composition has total alkalinity as CaCO₃ of about 2100 mg/L to about 2150 mg/L. In some embodiments, the alkaline water composition has total alkalinity as CaCO₃ of about 1100 mg/L. In some embodiments, the alkaline water composition has total alkalinity as CaCO₃ of about 1200 mg/L. In some embodiments, the alkaline water composition has total alkalinity as CaCO₃ of about 1300 mg/L. In some embodiments, the alkaline water composition has total alkalinity as CaCO₃ of about 1400 mg/L. In some embodiments, the alkaline water composition has total alkalinity as CaCO₃ of about 1500 mg/L. In some embodiments, the alkaline water composition has total alkalinity as CaCO₃ of about 1600 mg/L. In some embodiments, the alkaline water composition has total alkalinity as CaCO₃ of about 1700 mg/L. In some embodiments, the alkaline water composition has total alkalinity as CaCO₃ of about 1800 mg/L. In some embodiments, the alkaline water composition has total alkalinity as CaCO₃ of about 1900 mg/L. In some embodiments, the alkaline water composition has total alkalinity as CaCO₃ of about 2000 mg/L. In some embodiments, the alkaline water composition has total alkalinity as CaCO₃ of about 2100 mg/L. In some embodiments, the alkaline water composition has total alkalinity as CaCO₃ of about 2200 mg/L. In some embodiments, the alkaline water composition has total alkalinity as CaCO₃ of about 2300 mg/L. In some embodiments, the alkaline water composition has total alkalinity as CaCO₃ of about 2400 mg/L. In some embodiments, the alkaline water composition has total alkalinity as CaCO₃ of about 2500 mg/L. In some embodiments, the alkaline water composition has total alkalinity as CaCO₃ of about 2600 mg/L. In some embodiments, the alkaline water composition has total alkalinity as CaCO₃ of about 2700 mg/L. In some embodiments, the alkaline water composition has total alkalinity as CaCO₃ of about 2800 mg/L.

In some embodiments, the alkaline water composition has carbonate alkalinity as CaCO₃ of about 0 mg/L to about 100 mg/L. In some embodiments, the alkaline water composition has carbonate alkalinity as CaCO₃ of about 0 mg/L to about 75 mg/L. In some embodiments, the alkaline water composition has carbonate alkalinity as CaCO₃ of about 0 mg/L to about 50 mg/L. In some embodiments, the alkaline water composition has carbonate alkalinity as CaCO₃ of about 0 mg/L to about 25 mg/L. In some embodiments, the alkaline water composition has carbonate alkalinity as CaCO₃ of about 0 mg/L to about 20 mg/L. In some embodiments, the alkaline water composition has carbonate alkalinity as CaCO₃ of about 0 mg/L to about 15 mg/L. In some embodiments, the alkaline water composition has carbonate alkalinity as CaCO₃ of about 0 mg/L to about 10 mg/L. In some embodiments, the alkaline water composition has carbonate alkalinity as CaCO₃ of about 0 mg/L to about 5 mg/L. In some embodiments, the alkaline water composition has carbonate alkalinity as CaCO₃ of about 0 mg/L to about 4 mg/L. In some embodiments, the alkaline water composition has carbonate alkalinity as CaCO₃ of about 0 mg/L to about 3 mg/L. In some embodiments, the alkaline water composition has carbonate alkalinity as CaCO₃ of about 0 mg/L to about 2 mg/L. In some embodiments, the alkaline water composition has carbonate alkalinity as CaCO₃ of about 0 mg/L to about 1 mg/L. In some embodiments, the alkaline water composition has carbonate alkalinity as CaCO₃ of about 0 mg/L to about 0.5 mg/L. In some embodiments, the alkaline water composition has carbonate alkalinity as CaCO₃ of about 0.1 mg/L to about 5 mg/L. In some embodiments, the alkaline water composition has carbonate alkalinity as CaCO₃ of about 0.1 mg/L to about 4 mg/L. In some embodiments, the alkaline water composition has carbonate alkalinity as CaCO₃ of about 0.1 mg/L to about 3 mg/L. In some embodiments, the alkaline water composition has carbonate alkalinity as CaCO₃ of about 0.1 mg/L to about 2 mg/L. In some embodiments, the alkaline water composition has carbonate alkalinity as CaCO₃ of about 0.1 mg/L to about 1 mg/L. In some embodiments, the alkaline water composition has carbonate alkalinity as CaCO₃ of about 0.1 mg/L to about 0.5 mg/L. In some embodiments, the alkaline water composition has carbonate alkalinity as CaCO₃ of less than about 10 mg/L. In some embodiments, the alkaline water composition has carbonate alkalinity as CaCO₃ of less than about 5 mg/L. In some embodiments, the alkaline water composition has carbonate alkalinity as CaCO₃ of less than about 4 mg/L. In some embodiments, the alkaline water composition has carbonate alkalinity as CaCO₃ of less than about 3 mg/L. In some embodiments, the alkaline water composition has carbonate alkalinity as CaCO₃ of less than about 2 mg/L. In some embodiments, the alkaline water composition has carbonate alkalinity as CaCO₃ of less than about 1 mg/L. In some embodiments, the alkaline water composition has carbonate alkalinity as CaCO₃ of about 10 mg/L. In some embodiments, the alkaline water composition has carbonate alkalinity as CaCO₃ of about 5 mg/L. In some embodiments, the alkaline water composition has carbonate alkalinity as CaCO₃ of about 4 mg/L. In some embodiments, the alkaline water composition has carbonate alkalinity as CaCO₃ of about 3 mg/L. In some embodiments, the alkaline water composition has carbonate alkalinity as CaCO₃ of about 2 mg/L. In some embodiments, the alkaline water composition has carbonate alkalinity as CaCO₃ of about 1 mg/L.

In some embodiments, the alkaline water composition has carbonate alkalinity as CaCO₃ of about 750 mg/L to about 950 mg/L. In some embodiments, the alkaline water composition has carbonate alkalinity as CaCO₃ of about 800 mg/L to about 950 mg/L. In some embodiments, the alkaline water composition has carbonate alkalinity as CaCO₃ of about 800 mg/L to about 900 mg/L. In some embodiments, the alkaline water composition has carbonate alkalinity as CaCO₃ of about 850 mg/L to about 900 mg/L. In some embodiments, the alkaline water composition has carbonate alkalinity as CaCO₃ of about 750 mg/L. In some embodiments, the alkaline water composition has carbonate alkalinity as CaCO₃ of about 800 mg/L. In some embodiments, the alkaline water composition has carbonate alkalinity as CaCO₃ of about 850 mg/L. In some embodiments, the alkaline water composition has carbonate alkalinity as CaCO₃ of about 900 mg/L. In some embodiments, the alkaline water composition has carbonate alkalinity as CaCO₃ of about 950 mg/L. In some embodiments, the alkaline water composition has carbonate alkalinity as CaCO₃ of about 1000 mg/L.

In some embodiments, the alkaline water composition has bicarbonate alkalinity as CaCO₃ of about 1150 mg/L to about 2000 mg/L. In some embodiments, the alkaline water composition has bicarbonate alkalinity as CaCO₃ of about 1150 mg/L to about 1950 mg/L. In some embodiments, the alkaline water composition has bicarbonate alkalinity as CaCO₃ of about 1200 mg/L to about 1950 mg/L. In some embodiments, the alkaline water composition has bicarbonate alkalinity as CaCO₃ of about 1200 mg/L to about 1900 mg/L. In some embodiments, the alkaline water composition has bicarbonate alkalinity as CaCO₃ of about 1100 mg/L to about 1400 mg/L. In some embodiments, the alkaline water composition has bicarbonate alkalinity as CaCO₃ of about 1150 mg/L to about 1400 mg/L. In some embodiments, the alkaline water composition has bicarbonate alkalinity as CaCO₃ of about 1200 mg/L to about 1400 mg/L. In some embodiments, the alkaline water composition has bicarbonate alkalinity as CaCO₃ of about 1200 mg/L to about 1350 mg/L. In some embodiments, the alkaline water composition has bicarbonate alkalinity as CaCO₃ of about 1200 mg/L to about 1300 mg/L. In some embodiments, the alkaline water composition has bicarbonate alkalinity as CaCO₃ of about 1250 mg/L to about 1300 mg/L. In some embodiments, the alkaline water composition has bicarbonate alkalinity as CaCO₃ of about 1600 mg/L to about 2000 mg/L. In some embodiments, the alkaline water composition has bicarbonate alkalinity as CaCO₃ of about 1650 mg/L to about 2000 mg/L. In some embodiments, the alkaline water composition has bicarbonate alkalinity as CaCO₃ of about 1650 mg/L to about 1950 mg/L. In some embodiments, the alkaline water composition has bicarbonate alkalinity as CaCO₃ of about 1700 mg/L to about 1950 mg/L. In some embodiments, the alkaline water composition has bicarbonate alkalinity as CaCO₃ of about 1700 mg/L to about 1900 mg/L. In some embodiments, the alkaline water composition has bicarbonate alkalinity as CaCO₃ of about 1750 mg/L to about 1900 mg/L. In some embodiments, the alkaline water composition has bicarbonate alkalinity as CaCO₃ of about 1800 mg/L to about 1900 mg/L. In some embodiments, the alkaline water composition has bicarbonate alkalinity as CaCO₃ of about 1850 mg/L to about 1900 mg/L. In some embodiments, the alkaline water composition has bicarbonate alkalinity as CaCO₃ of about 1100 mg/L. In some embodiments, the alkaline water composition has bicarbonate alkalinity as CaCO₃ of about 1150 mg/L. In some embodiments, the alkaline water composition has bicarbonate alkalinity as CaCO₃ of about 1200 mg/L. In some embodiments, the alkaline water composition has bicarbonate alkalinity as CaCO₃ of about 1250 mg/L. In some embodiments, the alkaline water composition has bicarbonate alkalinity as CaCO₃ of about 1300 mg/L. In some embodiments, the alkaline water composition has bicarbonate alkalinity as CaCO₃ of about 1350 mg/L. In some embodiments, the alkaline water composition has bicarbonate alkalinity as CaCO₃ of about 1400 mg/L. In some embodiments, the alkaline water composition has bicarbonate alkalinity as CaCO₃ of about 1450 mg/L. In some embodiments, the alkaline water composition has bicarbonate alkalinity as CaCO₃ of about 1500 mg/L. In some embodiments, the alkaline water composition has bicarbonate alkalinity as CaCO₃ of about 1550 mg/L. In some embodiments, the alkaline water composition has bicarbonate alkalinity as CaCO₃ of about 1600 mg/L. In some embodiments, the alkaline water composition has bicarbonate alkalinity as CaCO₃ of about 1650 mg/L. In some embodiments, the alkaline water composition has bicarbonate alkalinity as CaCO₃ of about 1700 mg/L. In some embodiments, the alkaline water composition has bicarbonate alkalinity as CaCO₃ of about 1750 mg/L. In some embodiments, the alkaline water composition has bicarbonate alkalinity as CaCO₃ of about 1800 mg/L. In some embodiments, the alkaline water composition has bicarbonate alkalinity as CaCO₃ of about 1850 mg/L. In some embodiments, the alkaline water composition has bicarbonate alkalinity as CaCO₃ of about 1900 mg/L. In some embodiments, the alkaline water composition has bicarbonate alkalinity as CaCO₃ of about 1950 mg/L. In some embodiments, the alkaline water composition has bicarbonate alkalinity as CaCO₃ of about 2000 mg/L.

In some embodiments, the alkaline water composition has oxidation reduction potential of about −150 to about −25. In some embodiments, the alkaline water composition has oxidation reduction potential of about −150 to about −50. In some embodiments, the alkaline water composition has oxidation reduction potential of about −150 to about −75. In some embodiments, the alkaline water composition has oxidation reduction potential of about −150 to about −100. In some embodiments, the alkaline water composition has oxidation reduction potential of about −140 to about −100. In some embodiments, the alkaline water composition has oxidation reduction potential of about −140 to about −110. In some embodiments, the alkaline water composition has oxidation reduction potential of about −130 to about −110. In some embodiments, the alkaline water composition has oxidation reduction potential of about −125 to about 275. In some embodiments, the alkaline water composition has oxidation reduction potential of about −100 to about 275. In some embodiments, the alkaline water composition has oxidation reduction potential of about −100 to about 250. In some embodiments, the alkaline water composition has oxidation reduction potential of about −125 to about −25. In some embodiments, the alkaline water composition has oxidation reduction potential of about −125 to about −50. In some embodiments, the alkaline water composition has oxidation reduction potential of about −125 to about −75. In some embodiments, the alkaline water composition has oxidation reduction potential of about −100 to about −50. In some embodiments, the alkaline water composition has oxidation reduction potential of about −100 to about −75. In some embodiments, the alkaline water composition has oxidation reduction potential of about 150 to about 275. In some embodiments, the alkaline water composition has oxidation reduction potential of about 175 to about 275. In some embodiments, the alkaline water composition has oxidation reduction potential of about 175 to about 250. In some embodiments, the alkaline water composition has oxidation reduction potential of about 200 to about 250. In some embodiments, the alkaline water composition has oxidation reduction potential of about 200 to about 225. In some embodiments, the alkaline water composition has oxidation reduction potential of about −150. In some embodiments, the alkaline water composition has oxidation reduction potential of about −125. In some embodiments, the alkaline water composition has oxidation reduction potential of about −121. In some embodiments, the alkaline water composition has oxidation reduction potential of about −100. In some embodiments, the alkaline water composition has oxidation reduction potential of about −82. In some embodiments, the alkaline water composition has oxidation reduction potential of about −75. In some embodiments, the alkaline water composition has oxidation reduction potential of about −50. In some embodiments, the alkaline water composition has oxidation reduction potential of about −25. In some embodiments, the alkaline water composition has oxidation reduction potential of about 0. In some embodiments, the alkaline water composition has oxidation reduction potential of about 25. In some embodiments, the alkaline water composition has oxidation reduction potential of about 50. In some embodiments, the alkaline water composition has oxidation reduction potential of about 75. In some embodiments, the alkaline water composition has oxidation reduction potential of about 100. In some embodiments, the alkaline water composition has oxidation reduction potential of about 125. In some embodiments, the alkaline water composition has oxidation reduction potential of about 150. In some embodiments, the alkaline water composition has oxidation reduction potential of about 175. In some embodiments, the alkaline water composition has oxidation reduction potential of about 200. In some embodiments, the alkaline water composition has oxidation reduction potential of about 225. In some embodiments, the alkaline water composition has oxidation reduction potential of about 250. In some embodiments, the alkaline water composition has oxidation reduction potential of about 275. In some embodiments, the alkaline water composition has oxidation reduction potential of about 300.

In some embodiments, the alkaline water composition has electrical conductance of about 200 μmhos/cm to about 900 μmhos/cm. In some embodiments, the alkaline water composition has electrical conductance of about 200 μmhos/cm to about 700 μmhos/cm. In some embodiments, the alkaline water composition has electrical conductance of about 200 μmhos/cm to about 600 μmhos/cm. In some embodiments, the alkaline water composition has electrical conductance of about 200 μmhos/cm to about 650 μmhos/cm. In some embodiments, the alkaline water composition has electrical conductance of about 200 μmhos/cm to about 600 μmhos/cm. In some embodiments, the alkaline water composition has electrical conductance of about 200 μmhos/cm to about 550 μmhos/cm. In some embodiments, the alkaline water composition has electrical conductance of about 200 μmhos/cm to about 500 μmhos/cm. In some embodiments, the alkaline water composition has electrical conductance of about 200 μmhos/cm to about 450 μmhos/cm. In some embodiments, the alkaline water composition has electrical conductance of about 200 μmhos/cm to about 400 μmhos/cm. In some embodiments, the alkaline water composition has electrical conductance of about 200 μmhos/cm to about 350 μmhos/cm. In some embodiments, the alkaline water composition has electrical conductance of about 200 μmhos/cm to about 300 μmhos/cm. In some embodiments, the alkaline water composition has electrical conductance of about 3800 μmhos/cm to about 5750 μmhos/cm. In some embodiments, the alkaline water composition has electrical conductance of about 3850 μmhos/cm to about 5750 μmhos/cm. In some embodiments, the alkaline water composition has electrical conductance of about 3850 μmhos/cm to about 5700 μmhos/cm. In some embodiments, the alkaline water composition has electrical conductance of about 3900 μmhos/cm to about 5700 μmhos/cm. In some embodiments, the alkaline water composition has electrical conductance of about 3800 μmhos/cm to about 4100 μmhos/cm. In some embodiments, the alkaline water composition has electrical conductance of about 3800 μmhos/cm to about 4050 μmhos/cm. In some embodiments, the alkaline water composition has electrical conductance of about 3850 μmhos/cm to about 4050 μmhos/cm. In some embodiments, the alkaline water composition has electrical conductance of about 3850 μmhos/cm to about 4000 μmhos/cm. In some embodiments, the alkaline water composition has electrical conductance of about 3900 μmhos/cm to about 4000 μmhos/cm. In some embodiments, the alkaline water composition has electrical conductance of about 3900 μmhos/cm to about 3950 μmhos/cm. In some embodiments, the alkaline water composition has electrical conductance of about 5400 μmhos/cm to about 5800 μmhos/cm. In some embodiments, the alkaline water composition has electrical conductance of about 5450 μmhos/cm to about 5800 μmhos/cm. In some embodiments, the alkaline water composition has electrical conductance of about 5450 μmhos/cm to about 5750 μmhos/cm. In some embodiments, the alkaline water composition has electrical conductance of about 5500 μmhos/cm to about 5750 μmhos/cm. In some embodiments, the alkaline water composition has electrical conductance of about 5500 μmhos/cm to about 5700 μmhos/cm. In some embodiments, the alkaline water composition has electrical conductance of about 5550 μmhos/cm to about 5700 μmhos/cm. In some embodiments, the alkaline water composition has electrical conductance of about 5550 μmhos/cm to about 5650 μmhos/cm. In some embodiments, the alkaline water composition has electrical conductance of about 200 μmhos/cm. In some embodiments, the alkaline water composition has electrical conductance of about 250 μmhos/cm. In some embodiments, the alkaline water composition has electrical conductance of about 300 μmhos/cm. In some embodiments, the alkaline water composition has electrical conductance of about 350 μmhos/cm. In some embodiments, the alkaline water composition has electrical conductance of about 400 μmhos/cm. In some embodiments, the alkaline water composition has electrical conductance of about 450 μmhos/cm. In some embodiments, the alkaline water composition has electrical conductance of about 500 μmhos/cm. In some embodiments, the alkaline water composition has electrical conductance of about 550 μmhos/cm. In some embodiments, the alkaline water composition has electrical conductance of about 600 μmhos/cm. In some embodiments, the alkaline water composition has electrical conductance of about 650 μmhos/cm. In some embodiments, the alkaline water composition has electrical conductance of about 700 μmhos/cm. In some embodiments, the alkaline water composition has electrical conductance of about 3800 μmhos/cm. In some embodiments, the alkaline water composition has electrical conductance of about 3900 μmhos/cm. In some embodiments, the alkaline water composition has electrical conductance of about 4000 μmhos/cm. In some embodiments, the alkaline water composition has electrical conductance of about 4100 μmhos/cm. In some embodiments, the alkaline water composition has electrical conductance of about 4200 μmhos/cm. In some embodiments, the alkaline water composition has electrical conductance of about 4300 μmhos/cm. In some embodiments, the alkaline water composition has electrical conductance of about 4400 μmhos/cm. In some embodiments, the alkaline water composition has electrical conductance of about 4500 μmhos/cm. In some embodiments, the alkaline water composition has electrical conductance of about 4600 μmhos/cm. In some embodiments, the alkaline water composition has electrical conductance of about 4700 μmhos/cm. In some embodiments, the alkaline water composition has electrical conductance of about 4800 μmhos/cm. In some embodiments, the alkaline water composition has electrical conductance of about 4900 μmhos/cm. In some embodiments, the alkaline water composition has electrical conductance of about 5000 μmhos/cm. In some embodiments, the alkaline water composition has electrical conductance of about 5100 μmhos/cm. In some embodiments, the alkaline water composition has electrical conductance of about 5200 μmhos/cm. In some embodiments, the alkaline water composition has electrical conductance of about 5300 μmhos/cm. In some embodiments, the alkaline water composition has electrical conductance of about 5400 μmhos/cm. In some embodiments, the alkaline water composition has electrical conductance of about 5500 μmhos/cm. In some embodiments, the alkaline water composition has electrical conductance of about 5600 μmhos/cm. In some embodiments, the alkaline water composition has electrical conductance of about 5700 μmhos/cm. In some embodiments, the alkaline water composition has electrical conductance of about 5800 μmhos/cm.

In some embodiments, described herein is an alkaline water composition having the properties:

(a) about 250 mg/kg to about 500 mg/kg of sodium;

(b) about 1100 mg/kg to about 1300 mg/kg of sodium as sodium propionate;

(c) total dissolved solids of about 1650 mg/L to about 1750 mg/L;

(d) pH of about 13.0 to about 13.5;

(e) total alkalinity as CaCO₃ of about 2050 mg/L to about 2200 mg/L;

(f) carbonate alkalinity as CaCO₃ of about 750 mg/L to about 1000 mg/L;

(g) bicarbonate alkalinity as CaCO₃ of about 1200 mg/L to about 1400 mg/L;

(h) oxidation reduction potential of about −150 to about −25; and

(i) electrical conductance of about 200 μmhos/cm to about 300 μmhos/cm.

In some embodiments, described herein is an alkaline water composition having the properties:

(a) about 400 mg/kg of sodium;

(b) about 1200 mg/kg of sodium as sodium propionate;

(c) total dissolved solids of about 1650 mg/L to about 1750 mg/L;

(d) pH of about 13.2;

(e) total alkalinity as CaCO₃ of about 2120 mg/L;

(f) carbonate alkalinity as CaCO₃ of about 860 mg/L;

(g) bicarbonate alkalinity as CaCO₃ of about 1260 mg/L;

(h) oxidation reduction potential of about −121; and

(i) electrical conductance of about 254 μmhos/cm.

In some embodiments, described herein is an alkaline water composition having the properties:

(a) about 1300 mg/kg to about 1400 mg/kg of sodium;

(b) about 5600 mg/kg to about 5750 mg/kg of sodium as sodium propionate;

(c) total dissolved solids of about 5300 mg/L to about 5400 mg/L;

(d) pH of about 11.5 to about 12.5;

(e) total alkalinity as CaCO₃ of about 2050 mg/L to about 2200 mg/L;

(f) carbonate alkalinity as CaCO₃ of about 750 mg/L to about 1000 mg/L;

(g) bicarbonate alkalinity as CaCO₃ of about 1200 mg/L to about 1400 mg/L;

(h) oxidation reduction potential of about −100 to about −25; and

(i) electrical conductance of about 5500 μmhos/cm to about 5700 μmhos/cm.

In some embodiments, described herein is an alkaline water composition having the properties:

(a) about 1360 mg/kg of sodium;

(b) about 5685 mg/kg of sodium as sodium propionate;

(c) total dissolved solids of about 5350 mg/L;

(d) pH of about 12.2;

(e) total alkalinity as CaCO₃ of about 2120 mg/L;

(f) carbonate alkalinity as CaCO₃ of about 860 mg/L;

(g) bicarbonate alkalinity as CaCO₃ of about 1260 mg/L;

(h) oxidation reduction potential of about −82; and

(i) electrical conductance of about 5600 μmhos/cm.

In some embodiments, the alkaline water composition contains no buffers. In some embodiments, the alkaline water composition comprises pharmaceutical grade water. In some embodiments, the alkaline water composition comprises United States Pharmacopeia (USP) grade sodium propionate. In some embodiments, the alkaline water composition comprises European Pharmacopoeia (Ph. Eur.) grade sodium propionate. In some embodiments, the alkaline water composition comprises United States Pharmacopeia (USP) grade and European Pharmacopoeia (Ph. Eur.) grade sodium propionate.

In some embodiments, the alkaline water composition further comprises alcohol. In some embodiments, the alkaline water composition comprises USP-grade alcohol. In some embodiments, the alkaline water composition comprises about 10% to about 95% alcohol by volume. In some embodiments, the alkaline water composition comprises about 25% to about 90% alcohol by volume. In some embodiments, the alkaline water composition comprises about 85% alcohol and about 15% alkaline water by volume. In some embodiments, the alkaline water composition comprises about 75% alcohol and about 25% alkaline water by volume. In some embodiments, the alkaline water composition comprises about 65% alcohol and about 35% alkaline water by volume. In some embodiments, the alkaline water composition comprises about 50% alcohol and about 50% alkaline water by volume. In some embodiments, the alkaline water composition comprises about 40% alcohol and about 60% alkaline water by volume. In some embodiments, the alkaline water composition comprises about 30% alcohol and about 70% alkaline water by volume. In some embodiments, the alkaline water composition comprises about 25% alcohol and about 75% alkaline water by volume. In some embodiments, the alkaline water composition comprises about 20% alcohol and about 80% alkaline water by volume. In some embodiments, the alkaline water composition comprises about 10% alcohol and about 90% alkaline water by volume. In some embodiments, the alkaline water composition comprises alcohol, wherein the alcohol is selected from isopropanol, ethanol, and methanol. In some embodiments, the alkaline water composition comprises alcohol, wherein the alcohol is isopropanol. In some embodiments, the alkaline water composition comprises alcohol, wherein the alcohol is USP-grade isopropanol. In some embodiments, the alkaline water composition comprises alcohol, wherein the alcohol is ethanol. In some embodiments, the alkaline water composition comprises alcohol, wherein the alcohol is USP-grade ethanol. In some embodiments, the alkaline water composition comprises alcohol, wherein the alcohol is methanol. In some embodiments, the alkaline water composition comprises alcohol, wherein the alcohol is USP-grade methanol.

In some embodiments, the freezing point of the alkaline water composition is between about −80° C. to about −10° C. In some embodiments, the freezing point of the alkaline water composition is between about −70° C. to about −10° C. In some embodiments, the freezing point of the alkaline water composition is between about −60° C. to about −10° C. In some embodiments, the freezing point of the alkaline water composition is between about −50° C. to about −10° C. In some embodiments, the freezing point of the alkaline water composition is between about −50° C. to about −15° C. In some embodiments, the freezing point of the alkaline water composition is between about −40° C. to about −20° C. In some embodiments, the freezing point of the alkaline water composition is between about −40° C. to about −30° C.

In some embodiments of the alkaline water composition described herein, the alkaline water composition does not illicit a dermal irritation or sensitization response. In some embodiments of the alkaline water composition described herein, the alkaline water composition does not illicit a dermal irritation response. In some embodiments of the alkaline water composition described herein, the alkaline water composition does not illicit a sensitization response.

In some embodiments, the alkaline water composition is organic. In some embodiments, the alkaline water composition has a negative ionic charge. In some embodiments, the alkaline water composition is non-corrosive. In some embodiments, the alkaline water composition behaves like an anti-oxidant. In some embodiments, the alkaline water composition is an anti-oxidant. In some embodiments, the alkaline water composition is hypoallergenic. In some embodiments, the alkaline water composition is hypo-osmotic. In some embodiments, the alkaline water composition is safe for human oral consumption.

### Methods

In some embodiments, provided herein is a method of cleansing eyelashes in an individual in need thereof, comprising administering to the individual in need thereof an effective amount of an alkaline water composition described herein. In some embodiments, the cleansing is anti-microbial cleansing.

In some embodiments, provided herein is a method of cleansing the hair in an individual in need thereof, comprising administering to the individual in need thereof an effective amount of an alkaline water composition described herein. In some embodiments, provided herein is a method of cleansing the scalp in an individual in need thereof, comprising administering to the individual in need thereof an effective amount of an alkaline water composition described herein. In some embodiments, the cleansing is anti-microbial cleansing.

In some embodiments, provided herein is a method of cleansing the skin in an individual in need thereof, comprising administering to the individual in need thereof an effective amount of an alkaline water composition described herein. In some embodiments, provided herein is a method of cleansing the skin from sweat, dirt, or a combination thereof, in an individual in need thereof, comprising administering to the individual in need thereof an effective amount of an alkaline water composition described herein. In some embodiments, provided herein is a method of cleansing the skin from makeup, in an individual in need thereof, comprising administering to the individual in need thereof an effective amount of an alkaline water composition described herein. In some embodiments, the cleansing is anti-microbial cleansing.

In some embodiments, provided herein is a method of cleansing the teeth in an individual in need thereof, comprising administering to the individual in need thereof an effective amount of an alkaline water composition described herein. In some embodiments, the cleansing is anti-microbial cleansing. In some embodiments, provided herein is a method of whitening the teeth in an individual in need thereof, comprising administering to the individual in need thereof an effective amount of an alkaline water composition described herein.

In some embodiments, provided herein is a method of cleansing the palette in an individual in need thereof, comprising administering to the individual in need thereof an effective amount of an alkaline water composition described herein. In some embodiments, the cleansing is anti-microbial cleansing.

In some embodiments, provided herein is a method of hydrating the skin in an individual in need thereof, comprising administering to the individual in need thereof an effective amount of an alkaline water composition described herein.

In some embodiments, provided herein is a method of hydrating the eyelashes in an individual in need thereof, comprising administering to the individual in need thereof an effective amount of an alkaline water composition described herein.

In some embodiments, provided herein is a method of hydrating the hair in an individual in need thereof, comprising administering to the individual in need thereof an effective amount of an alkaline water composition described herein.

In some embodiments, provided herein is a method of hydrating the eyes in an individual in need thereof, comprising administering to the individual in need thereof an effective amount of an alkaline water composition described herein.

In some embodiments, provided herein is a method of hydrating fingernails and toenails in an individual in need thereof, comprising administering to the individual in need thereof an effective amount of an alkaline water composition described herein.

In some embodiments, provided herein is a method of hydrating the nasal passages in an individual in need thereof, comprising administering to the individual in need thereof an effective amount of an alkaline water composition described herein.

In some embodiments, provided herein is a method of treating burns in an individual in need thereof, comprising administering to the individual in need thereof an effective amount of an alkaline water composition described herein. In some embodiments, provided herein is a method of treating first degree burns in an individual in need thereof, comprising administering to the individual in need thereof an effective amount of an alkaline water composition described herein. In some embodiments, provided herein is a method of treating second degree burns in an individual in need thereof, comprising administering to the individual in need thereof an effective amount of an alkaline water composition described herein. In some embodiments, provided herein is a method of treating third degree burns in an individual in need thereof, comprising administering to the individual in need thereof an effective amount of an alkaline water composition described herein.

In some embodiments, provided herein is a method of treating sunburn in an individual in need thereof, comprising administering to the individual in need thereof an effective amount of an alkaline water composition described herein.

In some embodiments, provided herein is a method of reducing the effects of razor burn following shaving in an individual in need thereof, comprising administering to the individual in need thereof an effective amount of an alkaline water composition described herein.

In some embodiments, provided herein is a method of treating wounds in an individual in need thereof, comprising administering to the individual in need thereof an effective amount of an alkaline water composition described herein.

In some embodiments, provided herein is a method of treating pain in an individual in need thereof, comprising administering to the individual in need thereof an effective amount of an alkaline water composition described herein. In some embodiments, provided herein is a method of treating sore throat pain in an individual in need thereof, comprising administering to the individual in need thereof an effective amount of an alkaline water composition described herein.

In some embodiments, provided herein is a method of reducing the appearance of skin blemishes on the face in an individual in need thereof, comprising administering to the individual in need thereof an effective amount of an alkaline water composition described herein. In some embodiments, provided herein is a method of reducing the appearance of scars in an individual in need thereof, comprising administering to the individual in need thereof an effective amount of an alkaline water composition described herein. In some embodiments, provided herein is a method of reducing scarring after surgery or injury in an individual in need thereof, comprising administering to the individual in need thereof an effective amount of an alkaline water composition described herein.

In some embodiments, provided herein is a method of treating eczema in an individual in need thereof, comprising administering to the individual in need thereof an effective amount of an alkaline water composition described herein.

In some embodiments, provided herein is a method of treating inflammation in an individual in need thereof, comprising administering to the individual in need thereof an effective amount of an alkaline water composition described herein. In some embodiments, provided herein is a method of reducing inflammation in an individual in need thereof, comprising administering to the individual in need thereof an effective amount of an alkaline water composition described herein. In some embodiments, provided herein is a method of treating arthritis in an individual in need thereof, comprising administering to the individual in need thereof an effective amount of an alkaline water composition described herein.

### Application

In some embodiments, the alkaline water compositions described herein are applied to the individual as a liquid. In some embodiments, the alkaline water compositions described herein are applied to the individual as an aerosol. In some embodiments, the alkaline water compositions described herein are applied to the individual as a gel. In some embodiments, the alkaline water compositions described herein are applied to the individual as a cream. In some embodiments, the alkaline water compositions described herein are applied to the individual as an ointment. In some embodiments, the alkaline water compositions described herein are applied to the individual in a disposable wipe. In some embodiments, the alkaline water compositions described herein are applied to the individual as a serum. In some embodiments, the alkaline water compositions described herein are applied to the individual as a paste. In some embodiments, the alkaline water compositions described herein are applied to the individual in an injection.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The following examples provide illustrative methods for making exemplary alkaline water compositions. These examples are provided for illustrative purposes only and not to limit the scope of the claims provided herein.

### Example 1: Preparation of Pre-Mix Solution

A pre-mix solution was prepared using United States Pharmacopeia (USP) grade and European Pharmacopoeia (Ph. Eur.) grade sodium propionate and Ultra-High Purity (UHP) water. Accordingly, the pre-mix solution was prepared by mixing 20 grams of pharmaceutical-grade sodium propionate (USP and Ph. Eur.) per gallon of UHP water. Certificate of analysis for the pre-mix solution showed:

| Analyte | |
|---|---|
| Sodium, mg/kg | 1240 |
| Sodium, as Sodium Propionate, mg/kg | 5183 |
| Total Dissolved Solids, mg/L | 5250 |
| pH | 7.8 |
| Total Alkalinity as CaCO₃ mg/L | 1860 |
| Carbonate Alkalinity, as CaCO₃ mg/L | <2 |
| Bicarbonate Alkalinity, as CaCO₃ mg/L | 1860 |
| Oxidative Reduction Potential | 222 |
| Electrical Conductance, μmhos/cm | 3940 |

### Example 2: Preparation of Alkaline Water Composition

An initial stream of UHP water was heated to 150° F. The pre-mix solution from Example 1 was added to the initial stream to produce a feed stream. Residual undissolved sodium propionate in the pre-mix was removed from the feed stream through a first filter having a particle size of no more than 1 μm. The feed stream was then sterilized by heating the feed stream to a boiling temperature of at least 212° F. for at least three minutes to eliminate bacteria from the feed stream. The feed stream was electrolyzed by applying an electric potential of 30 amps, 5V to produce alkaline water adjacent to the cathode and acidic water adjacent to the anode. This results in two separate product streams, one being an alkaline solution (alkaline water composition) (pH between 10 and 12.5) and the other stream being an acidic solution (pH between 4 and 5.8). The two product streams are collected separately and analyzed. Certificate of analysis for the alkaline water composition showed:

| Analyte | |
|---|---|
| Sodium, mg/kg | 1360 |
| Sodium, as Sodium Propionate, mg/kg | 5685 |
| Total Dissolved Solids, mg/L | 5350 |
| pH | 12.2 |
| Total Alkalinity as CaCO₃ mg/L | 2120 |
| Carbonate Alkalinity, as CaCO₃ mg/L | 860 |
| Bicarbonate Alkalinity, as CaCO₃ mg/L | 1260 |
| Oxidative Reduction Potential | −82 |
| Electrical Conductance, μmhos/cm | 5600 |

### Example 3: Preparation of Alkaline Water Composition Containing Isopropanol

One stream of the alkaline water composition of Example 2 (1 part) and another stream of USP-grade isopropanol (IPA) (3 parts) are collected in an oxygen-free, sealed container at ambient temperature to afford an alkaline aqueous IPA composition with a total volume ratio of 75% IPA and 25% alkaline water. Analysis of the alkaline water composition containing IPA showed:

| Analyte | |
|---|---|
| Total Dissolved Solids, mg/L | 1710 |
| pH | 13.2 |
| Oxidative Reduction Potential | −121 |
| Electrical Conductance, μmhos/cm | 254 |

### Example 4: Analytical Methods

### Sodium

The amount of sodium in solution was determined using EPA 6010B, metals by ICP-OES.

### Sodium Propionate

The amount of sodium propionate in solution was determined based on the amount of sodium in solution and then stoichiometrically converted to sodium propionate with the factor 4.18.

### Total Dissolved Solids

Total dissolved solids in solution was determined via SM 2540 which involves gravimetric determination of total solids at 180° C. in a forced air oven.

pH

Samples were analyzed according to SM 4500 H-B 2000, pH in water by potentiometry. A pH meter is calibrated with appropriate buffers and the solution is analyzed. An ATC is used to correct for temperature fluctuations.

### Total Alkalinity, Bicarbonate, and Carbonate

Samples were analyzed according to SM 2320B, which involves titration with acid to pH 8.3 (phenolphthalein) and 4.5 (methyl orange). The stoichiometric relation of all these defines the sum of all the titratable bases in solution. Results are expressed as Calcium Carbonate.

### Oxidation Reduction Potential (ORP)

Samples were analyzed according to SM 2580, which involves reading the ORP using a Ag/AgCl reference electrode. Calibration was checked with ZoBell's solution (potassium ferrocyanide/potassium ferricyanide solution).

### Electrical Conductance

Samples were analyzed according to EPA 120.1, which involves using a calibrated meter to read the conductivity.

### Example 5: Assessment of Irritant Potential—Embryonic Hen's Egg Chorioallantoic Membrane Study

The study was based on the observation by a trained person of the irritant effects (hyperemia, hemorrhage, coagulation) occurring during the five minutes after application of test item to the chorioallantoic membrane (CAM) of embryonic hen's eggs on the tenth day of incubation. The irritant potential was scored according to a scale from 0 to 21. The test item was classified in one of the categories defined according to the mean score obtained.

### Preparation of the Eggs

Embryonic EOPS White Leghorn Hen's eggs (graded between 40 g and 75 g) were placed in a thermostated oven at 12° C.±1° C. out of light for at least 24 hours. Eggs were then put in an incubator under controlled conditions of temperature (37.8° C.±1° C.) and relative hygrometry (50 to 60%). During their 10-day incubation period, eggs were placed in an incubator with oscillating plates in upright position. On the 10^{th} day of incubation, eggs were taken out of the incubator one by one and candled with a lamp. The defective eggs (image did not correspond to the expected stage of development) were eliminated and the selected eggs were put in a holder. The shell of each selected egg was drilled with a lanceolate needle, opened and cut up at air pocket level and until the limits of the shell membrane. The whole surface of the shell membrane was moistened with a 0.9% sodium chloride solution, and warmed up at 37° C. Then, the egg was tilted to remove the excess 0.9% sodium chloride solution and the shell membrane was delicately removed with pliers to uncover the underlying CAM. Any egg whose CAM was damaged was immediately rejected.

### Application of the Test Item

The alkaline water composition of Example 2 and the alkaline water composition containing isopropanol of Example 3 were each separately tested on 4 different eggs. 300 μL of the composition of Example 3 was deposited on the CAM with a micropipette. After 20 seconds contact, the CAM was rinsed with 5 mL of sodium chloride isotonic solution with a syringe. The rinse liquid was removed by tilting the egg. The possible phenomena of irritation was observed during 5 minutes. The observation takin into account for the notation of the test item were performed visually. The accurate time of each phenomenon appearance was recorded. The phenomenon observed were defined as follows:
a) Hyperemia—capillaries that were not visible before the addition of the test item become visible whereas the visible capillaries dilate and become redder;
b) Hemorrhage—release of blood escaping from vessel and/or capillaries
c) Coagulation (opacity and/or thrombosis)—opacity: appearance of whole or part of the membrane, either of an opalescent veil possibly evolving to opacity or of direct opacity; thrombosis: rupture of blood flow in the vessels resulting in a segmented appearance.

### Expression and Interpretation of Test Results

The observed phenomena were qualified according to the table below in accordance with the rate of appearance:

| | Times | | |
|---|---|---|---|
| Phenomenon | t < 30 sec | 30 sec ≤ t ≤ 2 min | 2 min < t ≤ 5 min |
| Hyperemia | 5 | 3 | 1 |
| Hemorrhage | 7 | 5 | 3 |
| Coagulation | 9 | 7 | 5 |

Each phenomena observed was only counted once when it occurred. The score for each egg was the sum of the hyperemia, hemorrhage, and coagulation notes. The notation of the test item was the arithmetical mean of the 4 eggs, rounded up to one decimal. The following scale gave the irritant potential of the test item in the chorioallantoic membrane:

| Mean Score (MSc) | Classification |
|---|---|
| MSc < 1 | Non-irritant |
| 1 ≤ MSc < 5 | Slight irritant |
| 5 ≤ MSc <9 | Moderate irritant |
| MSc ≥ 9 | Irritant |

Test results showed that the Example 2 composition was a slight to moderate irritant (mean score 5±1.6) and the Example 3 composition was a non-irritant (mean score <1) in the chorioallantoic membrane test.

| Test Item | Classification |
|---|---|
| Example 2 composition | Slight/Moderate irritant |
| Example 3 composition | Non-irritant |

What is claimed is:

1. An alkaline water composition having the properties:
(a) about 200 mg/kg to about 1500 mg/kg of sodium;
(b) about 1000 mg/kg to about 5800 mg/kg of sodium as sodium propionate;
(c) total dissolved solids of about 1500 mg/L to about 5500 mg/L;
(d) pH of about 10.0 to about 13.5;
(e) total alkalinity as CaCO₃ of about 1100 mg/L to about 3000 mg/L;
(f) carbonate alkalinity as CaCO₃ of about 0 mg/L to about 1000 mg/L;
(g) bicarbonate alkalinity as CaCO₃ of about 1100 mg/L to about 2000 mg/L;
(h) oxidation reduction potential of about −150 to about 300; and
(i) electrical conductance of about 200 μmhos/cm to about 5800 μmhos/cm.

2. An alkaline water composition having the properties:
(a) about 1100 mg/kg to about 1500 mg/kg of sodium;
(b) about 5000 mg/kg to about 5800 mg/kg of sodium as sodium propionate;
(c) total dissolved solids of about 5000 mg/L to about 5500 mg/L;
(d) pH of about 10.0 to about 12.5;
(e) total alkalinity as CaCO₃ of about 1100 mg/L to about 3000 mg/L;
(f) carbonate alkalinity as CaCO₃ of about 0 mg/L to about 1000 mg/L;
(g) bicarbonate alkalinity as CaCO₃ of about 1100 mg/L to about 2000 mg/L;
(h) oxidation reduction potential of about −125 to about 300; and
(i) electrical conductance of about 3800 μmhos/cm to about 5800 μmhos/cm.

3. The alkaline water composition of claim 1 or claim 2 having about 200 mg/kg to about 550 mg/kg of sodium.

4. The alkaline water composition of claim 1 or claim 2 having about 250 mg/kg to about 500 mg/kg of sodium.

5. The alkaline water composition of claim 1 or claim 2 having about 300 mg/kg to about 450 mg/kg of sodium.

6. The alkaline water composition of claim 1 or claim 2 having about 1200 mg/kg to about 1400 mg/kg of sodium.

7. The alkaline water composition of claim 1 or claim 2 having about 1200 mg/kg to about 1300 mg/kg of sodium.

8. The alkaline water composition of claim 1 or claim 2 having about 1300 mg/kg to about 1400 mg/kg of sodium.

9. The alkaline water composition of any one of claims 1-8 having about 1000 mg/kg to about 1800 mg/kg of sodium as sodium propionate.

10. The alkaline water composition of any one of claims 1-8 having about 1000 mg/kg to about 1500 mg/kg of sodium as sodium propionate.

11. The alkaline water composition of any one of claims 1-8 having about 1100 mg/kg to about 1300 mg/kg of sodium as sodium propionate.

12. The alkaline water composition of any one of claims 1-8 having about 5100 mg/kg to about 5750 mg/kg of sodium as sodium propionate.

13. The alkaline water composition of any one of claims 1-8 having about 5100 mg/kg to about 5300 mg/kg of sodium as sodium propionate.

14. The alkaline water composition of any one of claims 1-8 having about 5600 mg/kg to about 5750 mg/kg of sodium as sodium propionate.

15. The alkaline water composition of any one of claims 1-14 having total dissolved solids of about 1500 mg/L to about 2000 mg/L.

16. The alkaline water composition of any one of claims 1-14 having total dissolved solids of about 1650 mg/L to about 1750 mg/L.

17. The alkaline water composition of any one of claims 1-14 having total dissolved solids of about 5200 mg/L to about 5400 mg/L.

18. The alkaline water composition of any one of claims 1-14 having total dissolved solids of about 5200 mg/L to about 5300 mg/L.

19. The alkaline water composition of any one of claims 1-14 having total dissolved solids of about 5300 mg/L to about 5400 mg/L.

20. The alkaline water composition of any one of claims 1-19 having a pH of about 11.5 to about 13.5.

21. The alkaline water composition of any one of claims 1-19 having a pH of about 12.5 to about 13.5.

22. The alkaline water composition of any one of claims 1-19 having a pH of about 13.0 to about 13.5.

23. The alkaline water composition of any one of claims 1-19 having a pH of about 10.5 to about 12.5.

24. The alkaline water composition of any one of claims 1-19 having a pH of about 11.5 to about 12.5.

25. The alkaline water composition of any one of claims 1-19 having a pH of about 12.0 to about 12.5.

26. The alkaline water composition of any one of claims 1-25 having total alkalinity as CaCO₃ of about 1500 mg/L to about 2600 mg/L.

27. The alkaline water composition of any one of claims 1-26 having total alkalinity as CaCO₃ of about 1700 mg/L to about 2200 mg/L.

28. The alkaline water composition of any one of claims 1-27 having total alkalinity as CaCO₃ of about 1700 mg/L to about 1950 mg/L.

29. The alkaline water composition of any one of claims 1-27 having total alkalinity as CaCO₃ of about 2050 mg/L to about 2200 mg/L.

30. The alkaline water composition of any one of claims 1-29 having carbonate alkalinity as CaCO₃ of about 0 mg/L to about 5 mg/L.

31. The alkaline water composition of any one of claims 1-29 having carbonate alkalinity as CaCO₃ of about 750 mg/L to about 1000 mg/L.

32. The alkaline water composition of any one of claims 1-31 having bicarbonate alkalinity as CaCO₃ of about 1200 mg/L to about 1900 mg/L.

33. The alkaline water composition of any one of claims 1-32 having bicarbonate alkalinity as CaCO₃ of about 1200 mg/L to about 1400 mg/L.

34. The alkaline water composition of any one of claims 1-32 having bicarbonate alkalinity as CaCO₃ of about 1700 mg/L to about 1900 mg/L.

35. The alkaline water composition of any one of claims 1-34 having oxidation reduction potential of about −150 to about −25.

36. The alkaline water composition of any one of claims 1-34 having oxidation reduction potential of about −150 to about −100.

37. The alkaline water composition of any one of claims 1-34 having oxidation reduction potential of about −100 to about 250.

38. The alkaline water composition of any one of claims 1-34 having oxidation reduction potential of about −100 to about −25.

39. The alkaline water composition of any one of claims 1-34 having oxidation reduction potential of about 200 to about 250.

40. The alkaline water composition of any one of claims 1-39 having electrical conductance of about 200 μmhos/cm to about 700 μmhos/cm.

41. The alkaline water composition of any one of claims 1-39 having electrical conductance of about 200 μmhos/cm to about 400 μmhos/cm.

42. The alkaline water composition of any one of claims 1-39 having electrical conductance of about 200 μmhos/cm to about 300 μmhos/cm.

43. The alkaline water composition of any one of claims 1-39 having electrical conductance of about 3900 μmhos/cm to about 5700 μmhos/cm.

44. The alkaline water composition of any one of claims 1-39 having electrical conductance of about 3900 μmhos/cm to about 4000 μmhos/cm.

45. The alkaline water composition of any one of claims 1-39 having electrical conductance of about 5500 μmhos/cm to about 5700 μmhos/cm.

46. The alkaline water composition of claim 1 or claim 2 having the properties:
(a) about 250 mg/kg to about 500 mg/kg of sodium;
(b) about 1100 mg/kg to about 1350 mg/kg of sodium as sodium propionate;
(c) total dissolved solids of about 1650 mg/L to about 1750 mg/L;
(d) pH of about 13.0 to about 13.5;
(e) total alkalinity as CaCO₃ of about 2050 mg/L to about 2200 mg/L;
(f) carbonate alkalinity as CaCO₃ of about 750 mg/L to about 1000 mg/L;
(g) bicarbonate alkalinity as CaCO₃ of about 1200 mg/L to about 1400 mg/L;
(h) oxidation reduction potential of about −150 to about −25; and
(i) electrical conductance of about 200 μmhos/cm to about 300 μmhos/cm.

47. The alkaline water composition of claim 1 having the properties:
(a) about 400 mg/kg of sodium;
(b) about 1200 mg/kg of sodium as sodium propionate;
(c) total dissolved solids of about 1650 mg/L to about 1750 mg/L;
(d) pH of about 13.2;
(e) total alkalinity as CaCO₃ of about 2120 mg/L;
(f) carbonate alkalinity as CaCO₃ of about 860 mg/L;
(g) bicarbonate alkalinity as CaCO₃ of about 1260 mg/L;
(h) oxidation reduction potential of about −121; and
(i) electrical conductance of about 254 μmhos/cm.

48. The alkaline water composition of claim 1 or claim 2 having the properties:
(a) about 1300 mg/kg to about 1400 mg/kg of sodium;
(b) about 5600 mg/kg to about 5750 mg/kg of sodium as sodium propionate;
(c) total dissolved solids of about 5300 mg/L to about 5400 mg/L;
(d) pH of about 11.5 to about 12.5;
(e) total alkalinity as CaCO₃ of about 2050 mg/L to about 2200 mg/L;
(f) carbonate alkalinity as CaCO₃ of about 750 mg/L to about 1000 mg/L;
(g) bicarbonate alkalinity as CaCO₃ of about 1200 mg/L to about 1400 mg/L;
(h) oxidation reduction potential of about −100 to about −25; and
(i) electrical conductance of about 5500 μmhos/cm to about 5700 μmhos/cm.

49. The alkaline water composition of claim 1 or claim 2 having the properties:
(a) about 1360 mg/kg of sodium;
(b) about 5685 mg/kg of sodium as sodium propionate;
(c) total dissolved solids of about 5350 mg/L;
(d) pH of about 12.2;
(e) total alkalinity as CaCO₃ of about 2120 mg/L;
(f) carbonate alkalinity as CaCO₃ of about 860 mg/L;
(g) bicarbonate alkalinity as CaCO₃ of about 1260 mg/L;
(h) oxidation reduction potential of about −82; and
(i) electrical conductance of about 5600 μmhos/cm.

50. The alkaline water composition of any one of claims 1-49, wherein the alkaline water composition contains no buffers.

51. The alkaline water composition of any one of claims 1-50, wherein the alkaline water composition comprises pharmaceutical grade water.

52. The alkaline water composition of any one of claims 1-51, wherein the alkaline water composition comprises United States Pharmacopeia (USP) grade sodium propionate.

53. The alkaline water composition of any one of claims 1-51, wherein the alkaline water composition comprises European Pharmacopoeia (Ph. Eur.) grade sodium propionate.

54. The alkaline water composition of any one of claims 1-51, wherein the alkaline water composition comprises United States Pharmacopeia (USP) grade and European Pharmacopoeia (Ph. Eur.) grade sodium propionate.

55. The alkaline water composition of any one of claims 1-54 further comprising alcohol.

56. The alkaline water composition of any one of claims 1-55 further comprising alcohol, wherein the composition comprises about 10% to about 95% alcohol by volume.

57. The alkaline water composition of any one of claims 1-56 further comprising alcohol, wherein the composition comprises about 25% to about 90% alcohol by volume.

58. The alkaline water composition of any one of claims 1-57 further comprising alcohol, wherein the composition comprises about 75% alcohol and about 25% alkaline water by volume.

59. The alkaline water composition of any one of claims 1-57 further comprising alcohol, wherein the composition comprises about 65% alcohol and about 35% alkaline water by volume.

60. The alkaline water composition of any one of claims 1-57 further comprising alcohol, wherein the composition comprises about 50% alcohol and about 50% alkaline water by volume.

61. The alkaline water composition of any one of claims 1-60 further comprising alcohol, wherein the alcohol is selected from isopropanol, ethanol, and methanol.

62. The alkaline water composition of any one of claims 1-61 further comprising alcohol, wherein the alcohol is isopropanol.

63. The alkaline water composition of any one of claims 1-61 further comprising alcohol, wherein the alcohol is ethanol.

64. The alkaline water composition of any one of claims 1-61 further comprising alcohol, wherein the alcohol is methanol.

65. The alkaline water composition of any one of claims 1-64, wherein the freezing point of the composition is between about −60° C. to about −10° C.

66. The alkaline water composition of any one of claims 1-64, wherein the freezing point of the composition is between about −50° C. to about −15° C.

67. The alkaline water composition of any one of claims 1-64, wherein the freezing point of the composition is between about −40° C. to about −20° C.

68. The alkaline water composition of any one of claims 1-64, wherein the freezing point of the composition is between about −40° C. to about −30° C.

69. The alkaline water composition of any one of claims 1-68, wherein the composition does not illicit a dermal irritation or sensitization response.

70. The alkaline water composition of any one of claims 1-69, wherein the alkaline water composition is organic.

71. The alkaline water composition of any one of claims 1-70, wherein the alkaline water composition has a negative ionic charge.

72. The alkaline water composition of any one of claims 1-71, wherein the alkaline water composition is an anti-oxidant.

73. The alkaline water composition of any one of claims 1-72, wherein the alkaline water composition is hypoallergenic.

74. The alkaline water composition of any one of claims 1-73, wherein the alkaline water composition is hypo-osmotic.

75. The alkaline water composition of any one of claims 1-74, wherein the alkaline water composition is safe for human oral consumption.

76. A method of cleansing eyelashes in an individual in need thereof, comprising administering to the individual in need thereof an effective amount of an alkaline water composition of any one of claims 1-75.

77. A method of cleansing the hair in an individual in need thereof, comprising administering to the individual in need thereof an effective amount of an alkaline water composition of any one of claims 1-75.

78. A method of cleansing the skin from sweat, dirt, or a combination thereof, in an individual in need thereof, comprising administering to the individual in need thereof an effective amount of an alkaline water composition of any one of claims 1-75.

79. The method of any one of claims 76-78, wherein the cleansing is anti-microbial cleansing.

80. A method of hydrating the skin in an individual in need thereof, comprising administering to the individual in need thereof an effective amount of an alkaline water composition of any one of claims 1-75.

81. A method of hydrating the eyelashes in an individual in need thereof, comprising administering to the individual in need thereof an effective amount of an alkaline water composition of any one of claims 1-75.

82. A method of hydrating the hair in an individual in need thereof, comprising administering to the individual in need thereof an effective amount of an alkaline water composition of any one of claims 1-75.

83. A method of treating sunburn in an individual in need thereof, comprising administering to the individual in need thereof an effective amount of an alkaline water composition of any one of claims 1-75.

84. A method of treating wounds in an individual in need thereof, comprising administering to the individual in need thereof an effective amount of an alkaline water composition of any one of claims 1-75.

85. A method of treating pain in an individual in need thereof, comprising administering to the individual in need thereof an effective amount of an alkaline water composition of any one of claims 1-75.

86. A method of treating inflammation in an individual in need thereof, comprising administering to the individual in need thereof an effective amount of an alkaline water composition of any one of claims 1-75.
